(19) United States
(12) Patent Application Publication
(10) Pub. No.: US 2019/0392720 A1
(43) Pub. Date: Dec. 26, 2019

(54) ASSESSING VISUAL PERFORMANCE

(71) Applicants: Alireza Farsi, Tehran (IR); Behrouz Abdoli, Tehran (IR); Mohsen Ghotbi, Esfeden (IR); Hesam Ramezanzade, Damghan (IR)

(72) Inventors: Alireza Farsi, Tehran (IR); Behrouz Abdoli, Tehran (IR); Mohsen Ghotbi, Esfeden (IR); Hesam Ramezanzade, Damghan (IR)

(21) Appl. No.: 16/016,553

(22) Filed: Jun. 23, 2018

(51) Int. Cl. G09B 5/02; G16H 40/20; G16H 20/30; A63B 69/00; G09B 19/16

(52) U.S. Cl. CPC G09B 5/02; G16H 40/20; A63B 69/0095; A63B 69/00; G09B 19/167; G16H 20/30

(57) ABSTRACT

One or more systems, devices and/or methods for assessing of at least one of coincidence-anticipation timing or motion perception of a user are provided. For example, a screen is controlled to display an interface comprising a first representation of an object moving from a starting point to an endpoint. The object is configured to reach the endpoint at a first time. An attempt to activate a response device when the object reaches the endpoint is monitored for. The attempt is detected at a second time by receiving a signal from the response device. An error of the attempt is generated based upon the first time and the second time. A report is generated comprising the error. The report may be representative of a coincidence-anticipation timing ability of the user.

### BACKGROUND

Many healthcare facilities, athletic organizations, companies, research centers, etc. may attempt to determine one or more abilities (e.g., a sports related ability, a driving ability, etc.) of an individual (e.g., an athlete, a patient, an employee, etc.), In some examples, various types of visual performance of the individual may be indicative of the one or more abilities of the individual.

### SUMMARY

In accordance with the present disclosure, one or more systems, devices and/or methods for assessing coincidence-anticipation timing and/or motion perception of a user are provided. In an example, a screen is controlled to display an interface comprising a first representation of a first object moving from a first starting point to a first endpoint. The first object is configured to reach the first endpoint at a first time. The first representation comprises the first object moving at a first speed, the first object moving with a first acceleration and the first object moving in a first direction. A first attempt to activate a response device when the first object reaches the first endpoint is monitored for. The first attempt is detected at a second time. The first attempt is detected by receiving a first signal from the response device. The screen is controlled to display the interface comprising a second representation of a second object moving from a second starting point to a second endpoint. The second object is configured to reach the second endpoint at a second time. The second representation comprises the second object moving at a second speed different from the first speed, the second object moving with a second acceleration different from the first acceleration and/or the second object moving in a second direction different from the first direction. A second attempt to activate the response device when the second object reaches the second endpoint is monitored for. The second attempt is detected at a fourth time. The second attempt is detected by receiving a second signal from the response device. A first error of the first attempt is generated based upon the first time and the second time. A second error of the second attempt is generated based upon the third time and the fourth time. A report is generated comprising the first error and the second error. The report may be representative of a coincidence-anticipation timing ability of the user.

In an example, a computing device is provided. The computing device is configured to control a screen to display an interface comprising a first representation of an object moving from a starting point to an endpoint. The object is configured to reach the endpoint at a first time. The computing device is configured to monitor for an attempt to activate a response device when the object reaches the endpoint. The computing device is configured to detect the attempt at a second time. The attempt is detected by receiving a signal from the response device. The computing device is configured to generate an error of the attempt based upon the first time and the second time. The computing device is configured to generate a report comprising the error. The report is representative of a coincidence-anticipation timing ability of the user. The computing device is configured to control a graphical user interface to display a second interface comprising the report and one or more selectable inputs. Each selectable input of the one or more selectable inputs corresponds to a parameter of a plurality of parameters of a second representation. The computing device is configured to receive, via the second interface, a request to present the second representation. The request comprises one or more selections of the one or more selectable inputs corresponding to the plurality of parameters. The computing device is configured to control the screen to display the interface comprising the second representation of a second object moving from a second starting point to a second endpoint.

In an example, a screen is controlled to display an interface comprising a first representation of an object moving from a starting point to an endpoint. The object is configured to reach the endpoint at a first time. An attempt to activate a response device when the object reaches the endpoint is monitored for. The attempt is detected at a second time. The attempt is detected by receiving a signal from the response device. An error of the attempt is generated based upon the first time and the second time. A report is generated comprising the error. The report may be representative of a coincidence-anticipation timing ability of the user.

### DESCRIPTION OF THE DRAWINGS

FIG. 1 is an illustration of an exemplary method for assessing coincidence-anticipation timing and/or motion perception of a user.

FIG. 2A is an illustration of an exemplary system for assessing coincidence-anticipation timing and/or motion perception of a user, wherein a screen is controlled to display an interface comprising a first representation of a first object moving from a first starting point to a first endpoint.

FIG. 2B is an illustration of an exemplary system for assessing coincidence-anticipation timing and/or motion perception of a user, wherein a screen is controlled to display an interface comprising a first representation of a first object moving from a first starting point to a first endpoint, wherein a first attempt may be detected at a second time.

FIG. 2C is an illustration of an exemplary system for assessing coincidence-anticipation timing and/or motion perception of a user, wherein a screen is controlled to display an interface comprising a second representation of a second object moving from a second starting point to a second endpoint.

FIG. 2D is an illustration of an exemplary system for assessing coincidence-anticipation timing and/or motion perception of a user, wherein a screen is controlled to display an interface comprising a second representation of a second object moving from a second starting point to a second endpoint, wherein a second attempt may be detected at a fourth time.

FIG. 2E is an illustration of an exemplary system for assessing coincidence-anticipation timing and/or motion perception of a user, wherein a screen is controlled to display an interface comprising a third representation of a third object moving from a third starting point to a third endpoint.

FIG. 2F is an illustration of an exemplary system for assessing coincidence-anticipation timing and/or motion perception of a user, wherein a screen is controlled to display an interface comprising a third representation of a third object moving from a third starting point to a third endpoint, wherein a third attempt may be detected at a sixth time.

FIG. 2G is an illustration of an exemplary system for assessing coincidence-anticipation timing and/or motion perception of a user, wherein a graphical user interface is controlled to display a second interface comprising a report and/or a selectable input.

FIG. 3A is an illustration of an exemplary system for configuring a scenario comprising a plurality of representations, wherein a graphical user interface is controlled to display an interface comprising a plurality of selectable inputs corresponding to the scenario.

FIG. 3B is an illustration of an exemplary system for configuring a scenario comprising a plurality of representations, wherein a graphical user interface is controlled to display a second interface comprising a second plurality of selectable inputs corresponding to a plurality of parameters of a representation.

FIG. 4A is an illustration of an exemplary system for assessing coincidence-anticipation timing and/or motion perception of a user, wherein the response device comprises a switch.

FIG. 4B is an illustration of an exemplary system for assessing coincidence-anticipation timing and/or motion perception of a user, wherein the response device is configured for one or more first contexts and/or the response device comprises a light transmitter and/or a light sensor.

FIG. 4C is an illustration of an exemplary system for assessing coincidence-anticipation timing and/or motion perception of a user, wherein the response device is configured for one or more second contexts and/or the response device comprises a light transmitter and/or a light sensor.

FIG. 4D is an illustration of an exemplary system for assessing coincidence-anticipation timing and/or motion perception of a user, wherein the response device is configured for one or more third contexts and/or the response device comprises a light transmitter and/or a light sensor.

FIG. 5 is an illustration of an exemplary computer-readable medium comprising processor-executable instructions, wherein the processor executable instructions may be configured to embody one or more of the provisions set forth herein.

FIG. 6 illustrates an exemplary computing environment wherein one or more of the provisions set forth herein may be implemented.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are illustrated in block diagram form in order to facilitate describing the claimed subject matter.

One or more systems, devices and/or techniques for assessing coincidence-anticipation timing and/or motion perception of a user are provided. Determining a coincidence-anticipation timing ability and/or a motion perception ability of an individual may be necessary for (e.g., and/or may facilitate) determining a sports-related ability, a driving ability, etc. of the individual and/or abilities of the individual for performing routine activities. For example, many healthcare facilities, athletic organizations, companies, research centers, etc. may attempt to determine a coincidence-anticipation timing ability and/or a motion perception ability of an individual (e.g., an athlete, a patient, an employee, etc.) and/or training the individual to improve the coincidence-anticipation timing ability and/or the motion perception ability. Alternatively and/or additionally, the healthcare facilities, athletic organizations, companies, research centers, etc. may attempt to determine the coincidence-anticipation timing ability and/or the motion perception ability in a specific context (e.g., a specific sport, a specific activity, etc.). However, it may be difficult to accurately and/or precisely assess the coincidence-anticipation ability and/or the motion perception ability of the individual and/or determine the coincidence-anticipation ability and/or the motion perception ability of the individual in the specific context.

For example, some methods, techniques and/or devices used may provide assessments to determine coincidence-anticipation timing abilities and/or motion perception abilities of individuals by activating a plurality of lights to represent an object moving from a starting point (e.g., a first light of the plurality of lights) to an endpoint (e.g., a second light of the plurality of lights). The plurality of lights may be fixed (e.g., and/or in fixed locations), and thus, the starting point, the endpoint, a direction of movement of the object, etc. may not change (e.g., locations) throughout different assessments. Further, the assessment may not be provided in more than one context. Thus, in accordance with one or more techniques presented herein, assessments to determine coincidence-anticipation timing abilities of individuals may be provided by controlling a screen to display an interface comprising a representation of an object moving from a starting point to an endpoint. Parameters of the representation, such as the speed of the object, an acceleration of the object, a direction of movement of the object, the starting point, the endpoint, a shape of the object, a color of the object, a color of a background of the interface, etc. may be set (e.g., and/or adjusted) based upon the assessment, a context (e.g., a sport, an activity, etc.), etc. For example, the assessment may comprise a plurality of representations, wherein parameters of each representation of the plurality of representations may (e.g., or may not) differ from each other.

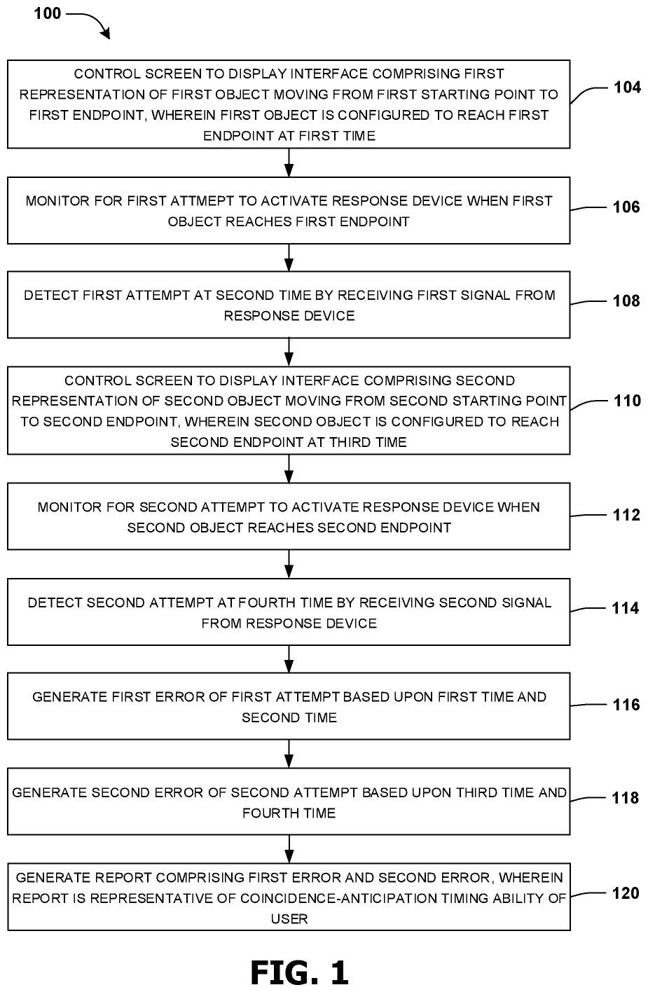

An embodiment for assessing coincidence-anticipation timing and/or motion perception of a user is illustrated by an example method 100 of FIG. 1. In some examples, a healthcare facility, an athletic organization, a company, a research center, a government agency, etc. may attempt to determine a coincidence-anticipation timing ability and/or a motion perception ability of the user. In some examples, the user may be a patient of the healthcare facility, an athlete associated with the athletic organization, an employee of the company, a test subject associated with the research center, a subject of a driving test administered by the government agency, etc. Accordingly, an administrator may assess coincidence-anticipation timing and/or motion perception of the user using one or more techniques and/or devices comprised herein. The administrator may be a healthcare worker, a coach, a researcher, a technician, an employee, a driving instructor, etc. of the healthcare facility, the athletic organization, the company, the research center, the government agency, etc. For example, the administrator may assess coincidence-anticipation timing and/or motion perception of the user in a context. The context may comprise a sport (e.g., tennis, soccer, basketball, volleyball, table tennis, track, etc.) and/or an activity (e.g., driving, walking outdoors, performing household functions, etc.). Alternatively and/or additionally, the administrator may train the user to improve the coincidence-anticipation timing ability and/or the motion perception ability using one or more techniques and/or devices comprised herein.

Accordingly, at 104, a screen may be controlled (e.g., by a device) to display an interface comprising a first representation of a first object moving from a first starting point to a first endpoint. The first object may be configured to reach the first endpoint at a first time. The first representation may comprise the first object moving at a first speed, the first object moving with a first acceleration and/or the first object moving in a first direction (e.g., and/or along a first path). In some examples, the device may be a computing device for assessing a visual performance of the user and/or training the user to improve the visual performance.

In some examples, the screen may be controlled by the device via one or more connections. For example, the screen may be coupled to the device via the one or more connections. The one or more connections may be wireless and/or wired connections. The screen may be a (e.g., computer) monitor, a television, a head-mounted display, a projection screen (e.g., used for displaying a projected image by a projector) and/or a different type of electronic display device. The user (e.g., and/or eyes of the user) may view the screen and/or observe the interface from a position relative to the screen (e.g., in front of the screen).

At 106, a first attempt to activate a response device when the first object reaches the first endpoint may be monitored for. For example, the user may be directed (e.g., by the administrator) to activate the response device when (e.g., at an instant that) the first object reaches (e.g., and/or coincides with) the first endpoint. Alternatively and/or additionally, the screen may be controlled to display instructions for the user to activate the response device when the first object reaches the first endpoint.

In some examples, one or more second connections of the device may be monitored to detect the first attempt. The one or more second connections may be wireless and/or wired connections. The response device may be coupled to the device via the one or more second connections. In some examples, the response device be positioned adjacent to (e.g., in front of, above, below, to a side of, etc.) the user. The response device may comprise a switch (e.g., a pushbutton, an on-off switch, etc.). The switch may be configured to transmit a first signal responsive to activation of the switch. The first signal may comprise an electronic signal (e.g., a pulse) and/or an electronic message indicating that the switch is activated.

Alternatively and/or additionally, the response device may comprise a light transmitter and/or a light sensor. The light transmitter may be configured to emit light (e.g., a laser beam and/or a different type of light) through a first location. The light sensor may be configured to monitor the light via the first location. For example, the light sensor may detect motion at the first location. In some examples, the response device may transmit the first signal (e.g., to the device) responsive to detecting motion at the first location. In some examples, the light transmitter and/or the light sensor may be positioned based upon the context of the first representation (e.g., and/or an assessment comprising the first representation).

At 108, the first attempt may be detected at a second time. The first attempt may be detected by receiving the first signal from the response device (e.g., at the second time). Responsive to detecting the first attempt, movement of the first object may be stopped (e.g., or the first object may continue moving along the first path). In some examples, the first time and/or the second time may be stored in a database of attempts stored on the device (e.g., and/or one or more servers connected to the device via a network connection).

At 110, the screen may be controlled (e.g., by the device) to display the interface comprising a second representation of a second object moving from a second starting point to a second endpoint. The second object may be configured to reach the second endpoint at a third time. The second representation may comprise the second object moving at a second speed, the second object moving with a second acceleration and/or the second object moving in a second direction (e.g., and/or along a second path). In some examples, the second speed of the second object may be different from the first speed of the first object, the second acceleration of the second object may be different from the first acceleration of the first object and/or the second direction (e.g., and/or the second path) of the second object may be different from the first direction (e.g., and/or the first path) of the first object.

At 112, a second attempt to activate the response device when the second object reaches the second endpoint may be monitored for. At 114, the second attempt may be detected at a fourth time. The second attempt may be detected by receiving a second signal from the response device (e.g., at the fourth time). Responsive to detecting the second attempt, movement of the second object may be stopped (e.g., or the second object may continue moving along the second path). In some examples, the third time and/or the fourth time may be stored in the database of attempts.

At 116, a first time error of the first attempt may be generated based upon the first time and the second time. For example, an (e.g., mathematical) operation may be performed on the first time and the second time to generate the first time error. In some examples, the first time error may comprise a first length of time between the first time and the second time. Alternatively and/or additionally, a first location of the first object at the second time (e.g., corresponding to the first attempt) may be determined. A first distance error may be generated based upon the first location and/or the first endpoint. For example, an (e.g., mathematical) operation may be performed on the first location and the first endpoint to generate the first distance error. In some examples, the first distance error may comprise a first distance between the first location and the first endpoint.

In some examples, the first time error and/or the first distance error may be generated responsive to receiving the first signal (e.g., corresponding to the first attempt). A graphical user interface (e.g., displayed on a second screen associated with the administrator) may be controlled to display a second interface comprising the first time error and/or the first distance error. Accordingly, the first time error and/or the first distance error may be presented to the administrator (e.g., and/or the user) responsive to the first attempt (e.g., and/or reception of the first signal).

At 118, a second time error of the second attempt may be generated based upon the third time and the fourth time. For example, an (e.g., mathematical) operation may be performed on the third time and the fourth time to generate the second time error. In some examples, the second time error may comprise a second length of time between the third time and the fourth time. Alternatively and/or additionally, a second location of the second object at the fourth time (e.g., corresponding to the second attempt) may be determined. A second distance error may be generated based upon the second location and/or the second endpoint. For example, an (e.g., mathematical) operation may be performed on the second location and the second endpoint to generate the second distance error. In some examples, the second distance error may comprise a second distance between the second location and the second endpoint.

In some examples, the second time error and/or the second distance error may be generated responsive to receiving the second signal (e.g., corresponding to the second attempt). The graphical user interface may be controlled to display the second interface comprising the second time error and/or the second distance error. Accordingly, the second time error and/or the second distance error may be presented to the administrator (e.g., and/or the user) responsive to the second attempt (e.g., and/or reception of the second signal).

In some examples, the first representation and/or the second representation may be comprised within a first set of representations. For example, each representation of the first set of representations may comprise an object moving from a starting point to an endpoint, wherein parameters of each representation of the first set of representations may be configured automatically and/or based upon a plurality of inputs received via the device.

For example, the graphical user interface may be controlled to display a third interface comprising a plurality of selectable inputs corresponding to a plurality of parameters of (e.g., each representation of) the first set of representations. Each selectable input of the plurality of selectable inputs may correspond to a parameter of the plurality of parameters. The plurality of parameters may comprise a set of parameters (e.g., a speed of an object, an acceleration of the object, a direction of movement of the object, a starting point, an endpoint, a shape of the object, a color of the object and/or a color of a background of the interface) for each representation of the first set of representations. For example, a first set of parameters of the first representation (e.g., of the first set of representations) may be different from a second set of parameters of the second representation (e.g., of the first set of representations). Alternatively and/or additionally, the first set of parameters may be the same as the second set of parameters.

The plurality of inputs (e.g., received via the device) may comprise an input corresponding to a time delay between each representation of the first set of representations. The time delay may be implemented between each representation of the first set of representations. For example, responsive to completion of the first representation (e.g., and/or responsive to receiving the first signal corresponding to the first attempt), the second representation may be displayed after a time corresponding to the time-delay. Alternatively and/or additionally, a set of time delays may be implemented, wherein a first time delay is implemented between the first representation and the second representation, a second time delay (e.g., different from the first time delay) is implemented between the second representation and a third representation (e.g., of the first set of representations), etc. The set of time delays may be (e.g., automatically and/or randomly) set by the device (e.g., and/or one or more servers connected to the device via a network) and/or each time delay of the set of time delays may be set via the plurality of inputs.

In some examples, a set of time errors (e.g., comprising the first time error and/or the second time error) may be determined based upon a set of attempts corresponding to the first set of representations and/or a set of signals (e.g., received from the response device) corresponding to the set of attempts. A set of distance errors (e.g., comprising the first distance error and/or the second distance error) may be determined based upon the set of attempts.

At 120, a report comprising the first time error and/or the second time error may be generated. The report may comprise the first distance error and/or the second distance error. Alternatively and/or additionally, the report may comprise the set of time errors (e.g., comprising the first time error and/or the second time error) and/or the set of distance errors (e.g., comprising the first distance error and/or the second distance error). The report may be representative of the coincidence-anticipation timing ability (e.g., and/or the motion response ability) of the user.

In some examples, a plurality of characteristics may be generated based upon the set of time errors and/or the set of distance errors. For example, the plurality of characteristics may comprise a first average time error and/or a first average distance error. For example, an (e.g., mathematical) operation may be performed on (e.g., absolute values of) the set of time errors to generate the first average time error. In some examples, the first average time error may comprise a first average of the set of time errors. Alternatively and/or additionally, an (e.g., mathematical) operation may be performed on (e.g., absolute values of) the set of distance errors to generate the first average distance error. In some examples, the first average distance error may comprise a second average of the set of distance errors.

In some examples, the plurality of characteristics may comprise one or more second average time errors corresponding to one or more portions of the set of time errors and/or one or more second average distance errors corresponding to one or more portions of the set of distance errors. The one or more second average time errors may comprise a second average time error corresponding to a first portion of the set of time errors. The first portion of the set of time errors may correspond to a first portion of the set of attempts. The first portion of the set of attempts may comprise a plurality of (e.g., initial) attempts of the set of attempts. Alternatively and/or additionally, the one or more second average distance errors may comprise a second average distance error corresponding to a first portion of the set of distance errors. The first portion of the set of distance errors may correspond to the first portion of the set of attempts.

In some examples, the one or more second average time errors may comprise a third average time error corresponding to a second portion of the set of time errors. The second portion of the set of time errors may correspond to a second portion of the set of attempts. The second portion of the set of attempts may comprise a plurality of (e.g., middle and/or last) attempts of the set of attempts. Alternatively and/or additionally, the one or more second average distance errors may comprise a third average distance error corresponding to a second portion of the set of distance errors. The second portion of the set of distance errors may correspond to the second portion of the set of attempts.

In some examples, each time error of the set of time errors may comprise a sign. For example, a time error of the set of time errors may comprise a negative number when a time of an (e.g., corresponding) attempt (e.g., or reception of a signal from the response device) is before a time that an object is configured to reach an endpoint. Alternatively and/or additionally, a time error of the set of time errors may comprise a positive number when a time of an (e.g., corresponding) attempt (e.g., or reception of a signal from the response device) is after a time that an object is configured to reach an endpoint.

Alternatively and/or additionally, each distance error of the set of distance errors may comprise a sign. For example, a distance error of the set of distance errors may comprise a negative number when a time of an (e.g., corresponding) attempt (e.g., or reception of a signal from the response device) is before a time that an object is configured to reach an endpoint. Alternatively and/or additionally, a distance error of the set of distance errors may comprise a positive number when a time of an (e.g., corresponding) attempt (e.g., or reception of a signal from the response device) is after a time that an object is configured to reach an endpoint.

In some examples, the plurality of characteristics may comprise a first maximum, a first minimum, a first mode, a first median, etc. of the set of time errors. Alternatively and/or additionally, the plurality of characteristics may comprise a second maximum, a second minimum, a second mode, a second median, etc. of the set of distance errors.

In some examples, the report may be generated as a file that can be accessed by using an external application, external software, etc. (e.g., a spreadsheet application, a database application, etc.). In some examples, the report may be presented via the graphical user interface. For example, the graphical user interface may be controlled to display a fourth interface comprising the report. The fourth interface may provide for browsing through, searching for, etc. the set of time errors, the set of distance errors, the plurality of characteristics, etc. The fourth interface may comprise a second plurality of selectable inputs corresponding to a second plurality of parameters of (e.g., each representation) of a second set of representations. The second plurality of parameters may comprise a set of parameters (e.g., a speed of an object, an acceleration of the object, a direction of movement of the object, a starting point, an endpoint, a shape of the object, a color of the object and/or a color of a background of the interface) for each representation of the second set of representations.

In some examples, the second plurality of parameters (e.g., and/or the second plurality of selectable inputs) may be selected (e.g., by the administrator and/or the user) based upon the report (e.g., the set of time errors, the set of distance errors, the plurality of characteristics, etc.). For example, the coincidence-anticipation ability (e.g., and/or the motion response ability) of the user may be represented by the report. The user may be assessed and/or trained by providing the second set of representations based upon the coincidence-anticipation ability of the user. For example, the report may comprise indications that the coincidence-anticipation ability (e.g., and/or the motion response ability) of the user may be at a first level indicating above-average performance. Accordingly, the second plurality of parameters (e.g., and/or the second plurality of selectable inputs) may be selected (e.g., by the administrator and/or the user) such that the second set of representations is at a second level of difficulty for the user. The second level of difficulty may be of higher difficulty than a first level of difficulty of the first set of representations. Alternatively and/or additionally, the report may comprise indications that the coincidence-anticipation ability (e.g., and/or the motion response ability) of the user may be at a second level indicating below-average performance. Accordingly, the second plurality of parameters (e.g., and/or the second plurality of selectable inputs) may be selected (e.g., by the administrator and/or the user) such that the second set of representations is at a third level of difficulty for the user. The third level of difficulty may be of lower difficulty than the first level of difficulty (e.g., of the first set of representations).

Alternatively and/or additionally, the second plurality of parameters may be selected (e.g., automatically) based upon the set of time errors, the set of distance errors, the plurality of characteristics, etc. corresponding to the first set of representations (e.g., by the device and/or by one or more servers connected to the device via a network). For example, the coincidence-anticipation ability of the user may be determined based upon the set of time errors, the set of distance errors, the plurality of characteristics, etc. The second plurality of parameters may be selected based upon the first set of representations and/or the coincidence-anticipation ability (e.g., and/or the motion response ability) of the user such that the second set of representations is at a fourth level of difficulty for the user.

The screen may be controlled (e.g., by the device) to display the interface comprising each representation of the second set of representations, consecutively. For example, the screen may be controlled to display the interface comprising a fourth representation (e.g., of the second set of representations) of a fourth object moving from a fourth starting point to a fourth endpoint. The fourth object may be configured to reach the fourth endpoint at a fifth time. One or more parameters (e.g., a speed of the fourth object, an acceleration of the fourth object, a direction of movement of the fourth object, the fourth starting point, the fourth endpoint, a shape of the fourth object, a color of the fourth object, a color of a background of the interface corresponding to the fourth representation, etc.) of the fourth representation may be based upon the second plurality of parameters.

A fourth attempt to activate the response device when the fourth object reaches the fourth endpoint may be monitored for. The fourth attempt may be detected at a sixth time. The fourth attempt may be detected by receiving a fourth signal from the response device (e.g., at the sixth time). In some examples, the fifth time and/or the sixth time may be stored in the database of attempts. In some examples, a fourth time error may be generated based upon the fifth time and the sixth time. The fourth time error may comprise a fourth length of time between the fifth time and the sixth time. Alternatively and/or additionally, a third location of the fourth object at the sixth time (e.g., corresponding to the fourth attempt) may be determined. A fourth distance error may be generated based upon the third location and/or the fourth endpoint. The fourth distance error may comprise a fourth distance between the third location and the fourth endpoint.

In some examples, a second set of time errors (e.g., comprising the fourth time error) may be determined based upon a second set of attempts corresponding to the second set of representations and/or a second set of signals (e.g., received from the response device) corresponding to the second set of attempts. A second set of distance errors (e.g., comprising the fourth distance error) may be determined based upon the second set of attempts. A second report comprising the second set of time errors and/or the second set of distance errors may be generated. The second report may comprise the set of time errors and/or the set of distance errors (e.g., corresponding to the first set of representations). A second plurality of characteristics may be generated based upon the second set of time errors and/or the second set of distance errors. The second plurality of characteristics (e.g., and/or the plurality of characteristics corresponding to the first set of representations) may be comprised within the second report.

In some examples, a treatment schedule for the user may be developed by a treatment unit (e.g., of the device, of one or more servers connected to the device by a network, etc.) based upon the report (e.g., and/or the second report). Alternatively and/or additionally, the treatment schedule may be developed by the administrator (e.g., a physician, a medical specialist, etc.) based upon the report (e.g., and/or the second report). For example, the user may be a patient undergoing treatment for one or more issues. The one or more issues may comprise visual performance-related issues (e.g., such as coincidence-anticipation timing, motion perception, reflex abilities, etc.). The treatment schedule may comprise a schedule for training activities for treating the user and/or improving visual performance of the user. The various training activities may be administered to the user via the device, the response device and/or the screen. The treatment schedule may comprise a second schedule for assessments for identifying any improvement in the visual performance of the user. The assessments may be administered to the user via the device, the response device and/or the screen. The treatment schedule may comprise a third schedule for other activities (e.g., training activities, assessments, tasks, examinations, lessons, workouts, etc. for the treatment of the user).

Alternatively and/or additionally, a training schedule for the user may be developed by a training unit (e.g., of the device, of one or more servers connected to the device by a network, etc.) based upon the report (e.g., and/or the second report). Alternatively and/or additionally, the training schedule may be developed by the administrator (e.g., a trainer, a coach, an athletic specialist, etc.). For example, the user may be an athlete seeking to improve sports-related abilities. For example, the training schedule may comprise a fourth schedule for various training activities corresponding to one or more contexts associated with the sports-related abilities for improving the sports-related abilities (e.g., that the user is seeking to improve). The training schedule may comprise a fifth schedule for assessments for identifying any improvement in the sports-related abilities. The training schedule may comprise a sixth schedule for other activities (e.g., sports exercises, drills, etc.) for training the user.

Alternatively and/or additionally, the user (e.g., an athlete) may be selected from a plurality of athletes for placement in a sports team, for placement in a (e.g., certain) position of the sports team, for placement in a competition, etc. by a placement unit (e.g., (e.g., of the device, of one or more servers connected to the device by a network, etc.) based upon the report (e.g., and/or the second report). Alternatively and/or additionally, the user may be selected from the plurality of athletes by the administrator (e.g., a trainer, a coach, an athletic specialist, etc.) based upon the report (e.g., and/or the second report).

Alternatively and/or additionally, a driving test result corresponding to a driving test of the user (e.g., a subject of the driving test) may be determined by a driving test unit (e.g., of the device, of one or more servers connected to the device by a network, etc.) based upon the report (e.g., and/or the second report). The driving test result may comprise an indication that a visual performance of the user is above or below a visual performance threshold. For example, responsive to the visual performance of the user being above the visual performance threshold, an approval corresponding to the driving test may be transmitted to a second device associated with the administrator (e.g., a driving instructor, a government employee tasked with administering driving tests, etc.). Responsive to the visual performance of the user being below the visual performance threshold, a disapproval corresponding to the driving test may be transmitted to the second device. Alternatively and/or additionally, the report may comprise the driving test result. Alternatively and/or additionally, the driving test result may be determined by the administrator based upon the report (e.g., and/or the second report).

Alternatively and/or additionally, motor-vehicle settings of a motor vehicle may be set and/or adjusted by a motor vehicle unit (e.g., of the device, of one or more servers connected to the device by a network, of a third device associated with the motor vehicle, etc.) based upon the report (e.g., and/or the second report). For example, the report (e.g., and/or the second report) may be transmitted to the third device (e.g., associated with the motor vehicle). The settings of the motor vehicle may be set and/or adjusted by the third device based upon the report (e.g., and/or the second report).

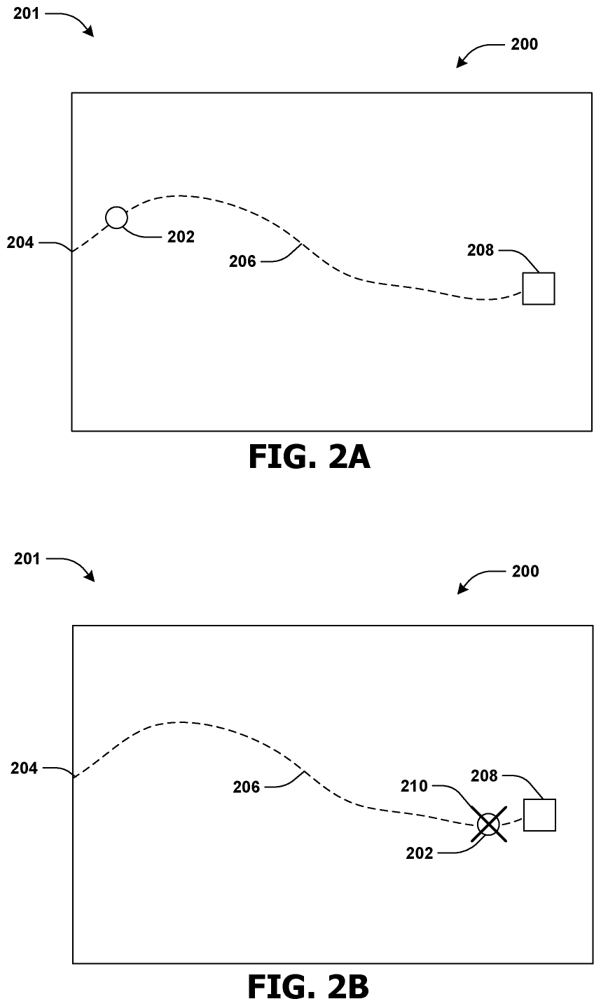

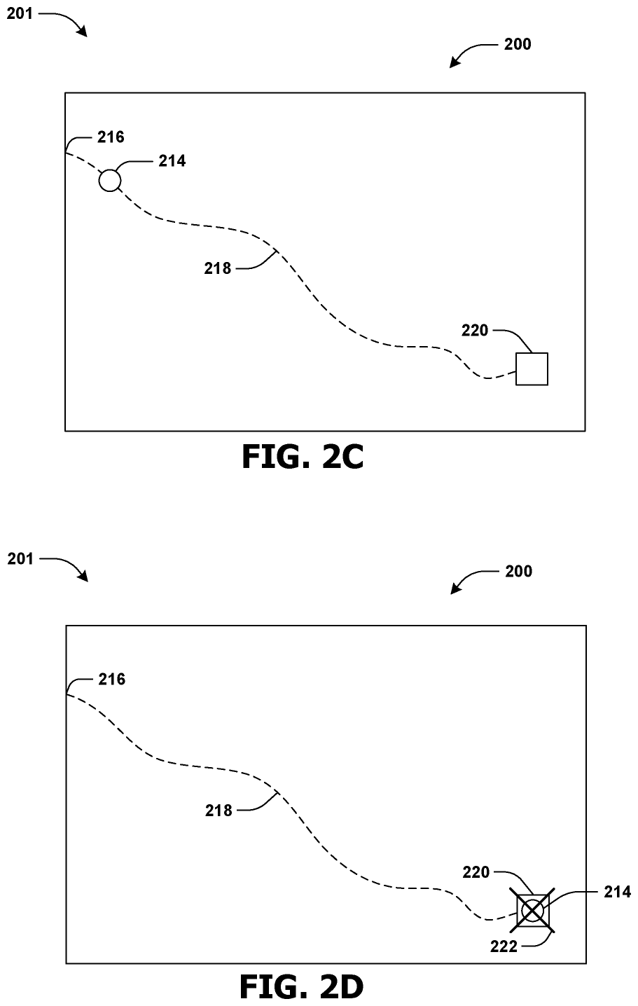

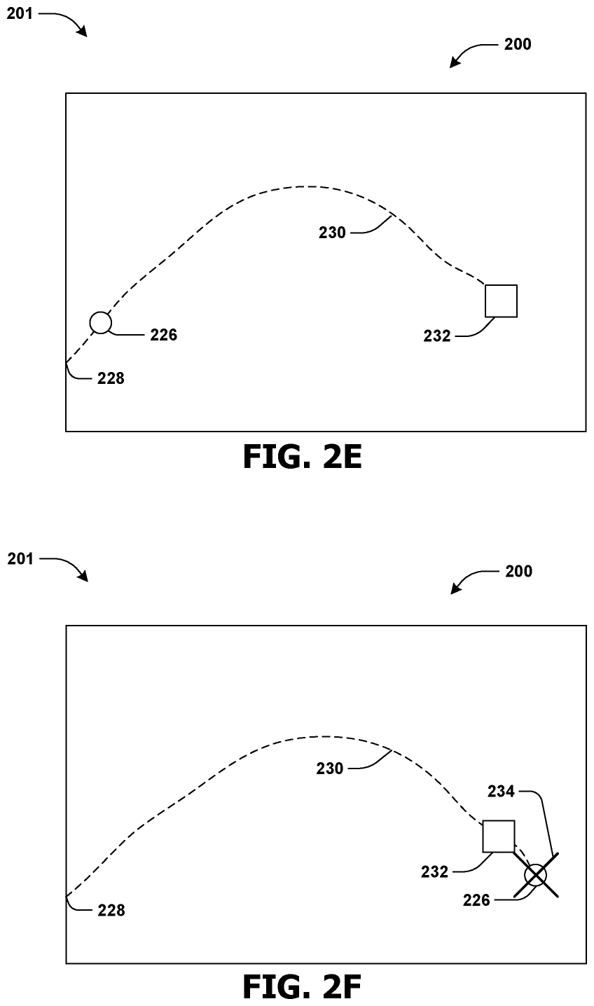

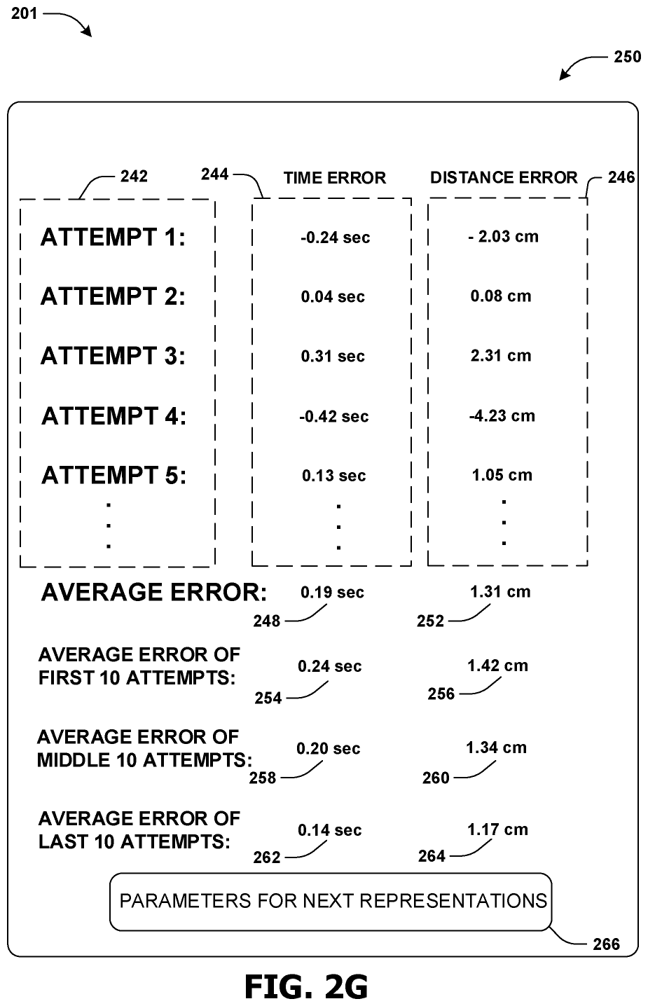

FIGS. 2A-2G illustrate examples of a system 201 for assessing coincidence-anticipation timing and/or motion perception of a user. A screen 200 may be a (e.g., computer) monitor, a television, a head-mounted display, a projection screen (e.g., used for displaying a projected image by a projector) and/or a different type of electronic display device. FIGS. 2A-2B illustrate the screen 200 being controlled (e.g., by a device of the system 201) to display an interface comprising a first representation of a first object 202 moving from a first starting point 204 to a first endpoint 208. The first object 202 may be configured to reach the first endpoint 208 at a first time. The first representation may comprise the first object 202 moving at a first speed, the first object 202 moving with a first acceleration and/or the first object 202 moving along a first path 206 (e.g., and/or in a first direction). In some examples, the first path 206 may be displayed (e.g., in the first representation). Alternatively and/or additionally, the first path 206 may not be displayed.

A first attempt to activate a response device when the first object 202 reaches the first endpoint 208 may be monitored for. The first attempt may be detected at a second time. The first attempt may be detected by receiving a first signal from the response device (e.g., at the second time). A first time error of the first attempt may be generated based upon the first time and the second time. For example, an (e.g., mathematical) operation may be performed on the first time and the second time to generate the first time error. In some examples, the first time error may comprise a first length of time between the first time and the second time.

A first location 210 of the first object 202 at the second time (e.g., corresponding to the first attempt) may be determined. A first distance error may be generated based upon the first location 210 and/or the first endpoint 208. For example, an (e.g., mathematical) operation may be performed on the first location 210 and the first endpoint 208 to generate the first distance error. In some examples, the first distance error may comprise a first distance between the first location 210 and the first endpoint 208.

FIGS. 2C-2D illustrate the screen 200 being controlled (e.g., by the device of the system 201) to display the interface comprising a second representation of a second object 214 moving from a second starting point 216 to a second endpoint 220. The second object 214 may be configured to reach the second endpoint 220 at a third time. The second representation may comprise the second object 214 moving at a second speed, the second object 214 moving with a second acceleration and/or the second object 214 moving along a second path 218 (e.g., and/or in a second direction). In some examples, the second path 218 may be displayed (e.g., in the second representation). Alternatively and/or additionally, the second path 218 may not be displayed.

A second attempt to activate the response device when the second object 214 reaches the second endpoint 220 may be monitored for. The second attempt may be detected at a fourth time. The second attempt may be detected by receiving a second signal from the response device (e.g., at the fourth time). A second time error of the second attempt may be generated based upon the third time and the fourth time. For example, an (e.g., mathematical) operation may be performed on the third time and the fourth time to generate the second time error. In some examples, the second time error may comprise a second length of time between the third time and the fourth time.

A second location 222 of the second object 214 at the fourth time (e.g., corresponding to the second attempt) may be determined. A second distance error may be generated based upon the second location 222 and/or the second endpoint 220. For example, an (e.g., mathematical) operation may be performed on the second location 222 and the second endpoint 220 to generate the second distance error. In some examples, the second distance error may comprise a second distance between the second location 222 and the second endpoint 220.

FIGS. 2E-2F illustrate the screen 200 being controlled (e.g., by the device of the system 201) to display the interface comprising a third representation of a third object 226 moving from a third starting point 228 to a third endpoint 232. The third object 226 may be configured to reach the third endpoint 232 at a fifth time. The third representation may comprise the third object 226 moving at a third speed, the third object 226 moving with a third acceleration and/or the third object 226 moving along a third path 230 (e.g., and/or in a third direction). In some examples, the third path 230 may be displayed (e.g., in the third representation). Alternatively and/or additionally, the third path 230 may not be displayed.

A third attempt to activate the response device when the third object 226 reaches the third endpoint 232 may be monitored for. The third attempt may be detected at a sixth time. The third attempt may be detected by receiving a third signal from the response device (e.g., at the sixth time). A third time error of the third attempt may be generated based upon the fifth time and the sixth time. For example, an (e.g., mathematical) operation may be performed on the fifth time and the sixth time to generate the third time error. In some examples, the third time error may comprise a third length of time between the fifth time and the sixth time.

A third location 234 of the third object 226 at the sixth time (e.g., corresponding to the third attempt) may be determined. A third distance error may be generated based upon the third location 234 and/or the third endpoint 232. For example, an (e.g., mathematical) operation may be performed on the third location 234 and the third endpoint 232 to generate the third distance error. In some examples, the third distance error may comprise a third distance between the third location 234 and the third endpoint 232.

In some examples, the first representation, the second representation and/or the third representation may be comprised within a first set of representations. For example, each representation of the first set of representations may comprise an object moving from a starting point to an endpoint, wherein parameters of each representation of the first set of representations may be configured automatically and/or based upon a plurality of inputs received via the device.

In some examples, the first set of representations may be a part of an assessment to determine a coincidence-anticipation timing ability and/or a motion perception ability of a user. Alternatively and/or additionally, the first set of representations may be a part of a training activity to improve the coincidence-anticipation ability and/or the motion perception ability of the user.

FIG. 2G illustrates a graphical user interface 250 being controlled (e.g., by the device of the system 201) to display a second interface comprising a report and/or a selectable input 266. In some examples, a set of time errors 244 (e.g., comprising the first time error, the second time error and/or the third time error) may be generated based upon a set of attempts 242 corresponding to the first set of representations and/or a set of signals (e.g., received from the response device) corresponding to the set of attempts 242. A set of distance errors 246 (e.g., comprising the first distance error, the second distance error and/or the third distance error) may be generated based upon the set of attempts 242. The report (e.g., displayed via the graphical user interface 250) may comprise the set of time errors 244 and/or the set of distance errors 246.

In some examples, a plurality of characteristics may be generated based upon the set of time errors 244 and/or the set of distance errors 246. The report may comprise the plurality of characteristics. For example, the plurality of characteristics may comprise a first average time error 248 and/or a first average distance error 252. For example, an (e.g., mathematical) operation may be performed on (e.g., absolute values of) the set of time errors 244 to generate the first average time error 248. In some examples, the first average time error 248 may comprise a first average of the set of time errors 244. Alternatively and/or additionally, an (e.g., mathematical) operation may be performed on (e.g., absolute values of) the set of distance errors 246 to generate the first average distance error 252. In some examples, the first average distance error 252 may comprise a second average of the set of distance errors 246.

In some examples, the plurality of characteristics may comprise a plurality of average time errors corresponding to a plurality of portions of the set of time errors 244. Alternatively and/or additionally, the plurality of characteristics may comprise a plurality of average distance errors corresponding to a plurality of portions of the set of distance errors 246.

For example, the plurality of average time errors may comprise a second average time error 254 corresponding to a first portion of the set of time errors 244. An (e.g., mathematical) operation may be performed on (e.g., absolute values of) the first portion of the set of time errors 244. The second average time error 254 may comprise a third average of the first portion of the set of time errors 244. The first portion of the set of time errors 244 may correspond to a first portion of the set of attempts 242. For example, the first portion of the set of attempts 242 may comprise (e.g., 10) initial attempts of the set of attempts 242.

The plurality of average time errors may comprise a third average time error 258 corresponding to a second portion of the set of time errors 244. An (e.g., mathematical) operation may be performed on (e.g., absolute values of) the second portion of the set of time errors 244. The third average time error 258 may comprise a fourth average of the second portion of the set of time errors 244. The second portion of the set of time errors 244 may correspond to a second portion of the set of attempts 242. For example, the second portion of the set of attempts 242 may comprise (e.g., 10) middle attempts of the set of attempts 242 (e.g., after the 10 initial attempts of the first portion of the set of attempts 242).

The plurality of average time errors may comprise a fourth average time error 262 corresponding to a third portion of the set of time errors 244. An (e.g., mathematical) operation may be performed on (e.g., absolute values of) the third portion of the set of time errors 244. The fourth average time error 262 may comprise a fifth average of the third portion of the set of time errors 244. The third portion of the set of time errors 244 may correspond to a third portion of the set of attempts 242. For example, the third portion of the set of attempts 242 may comprise (e.g., 10) last attempts of the set of attempts 242 (e.g., after the 10 middle attempts of the second portion of the set of attempts 242).

The plurality of average distance errors may comprise a second average distance error 256 corresponding to a first portion of the set of distance errors 246. An (e.g., mathematical) operation may be performed on (e.g., absolute values of) the first portion of the set of distance errors 246. The second average distance error 256 may comprise a sixth average of the first portion of the set of distance errors 246. The first portion of the set of distance errors 246 may correspond to the first portion of the set of attempts 242.

The plurality of average distance errors may comprise a third average distance error 260 corresponding to a second portion of the set of distance errors 246. An (e.g., mathematical) operation may be performed on (e.g., absolute values of) the second portion of the set of distance errors 246. The third average distance error 260 may comprise a seventh average of the second portion of the set of distance errors 246. The second portion of the set of distance errors 246 may correspond to the second portion of the set of attempts 242.

The plurality of average distance errors may comprise a fourth average distance error 264 corresponding to a third portion of the set of distance errors 246. An (e.g., mathematical) operation may be performed on (e.g., absolute values of) the third portion of the set of distance errors 246. The fourth average distance error 264 may comprise an eighth average of the third portion of the set of distance errors 246. The third portion of the set of distance errors 246 may correspond to the third portion of the set of attempts 242.

In some examples, the second interface may provide for browsing through, searching for, etc. the set of time errors 244, the set of distance errors 246, the plurality of characteristics, etc. The selectable input 266 may correspond to a plurality of parameters of a second set of representations (e.g., of the assessment and/or the training activity). For example, responsive to (e.g., receiving) a selection of the selectable input 266, a plurality of selectable inputs corresponding to the plurality of parameters may be displayed (e.g., via the second interface).

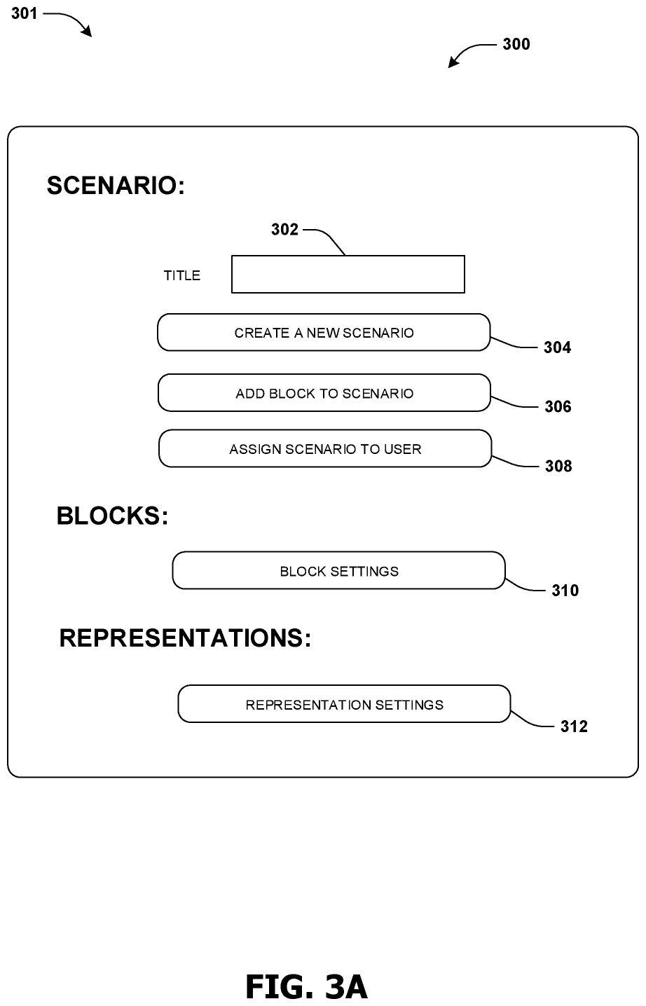

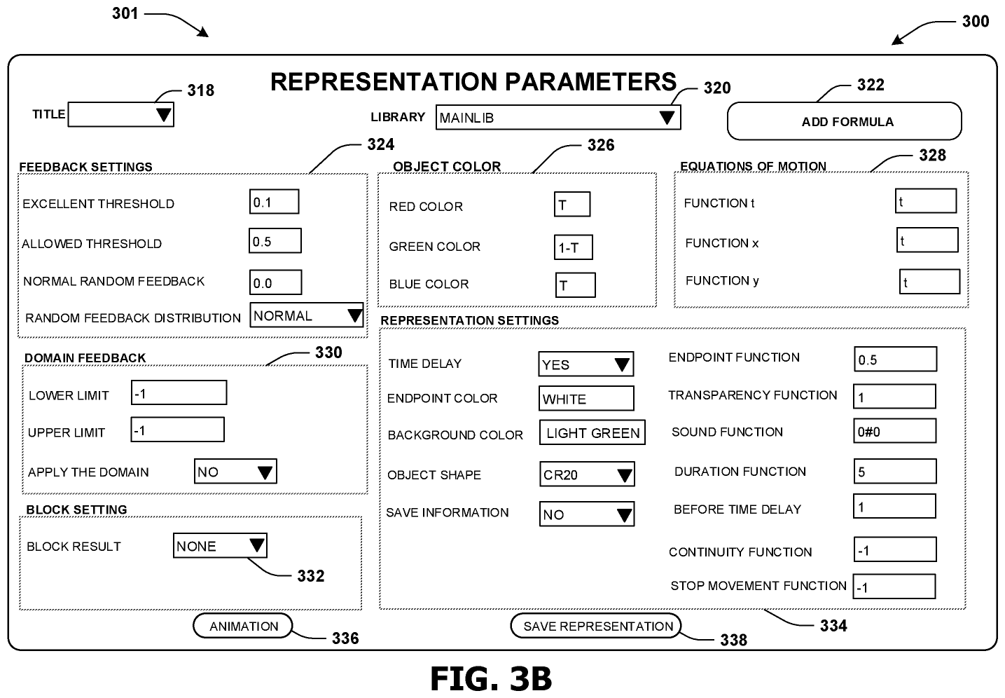

FIGS. 3A-3B illustrate examples of a system 301 for configuring a scenario comprising a plurality of representations. The scenario may be an assessment to determine a coincidence-anticipation timing ability and/or a motion perception ability of a user. Alternatively and/or additionally, the scenario may be a training activity to improve the coincidence-anticipation ability and/or the motion perception ability of the user.

FIG. 3A illustrates a graphical user interface 300 being controlled (e.g., by a device of the system 301) to display an interface comprising a plurality of selectable inputs corresponding to the scenario. For example, the graphical user interface 300 may be displayed (e.g., to an administrator associated with the assessment and/or the training activity). For example, the plurality of selectable inputs may comprise a first selectable input 302 “TITLE” corresponding to a title of the scenario. For example, responsive to a selection of the first selectable input 302 (e.g., by the administrator and/or a user associated with the assessment and/or the training activity), a title name of the title may be inputted (e.g., via a keyboard, a conversational interface, etc.). The plurality of selectable inputs may comprise a second selectable input 304 “CREATE A NEW SCENARIO”. Responsive to a selection of the second selectable input 304, the scenario may be recorded and/or stored (e.g., under the title name) in a database of scenarios. The database of scenarios may be stored on the device (e.g., and/or on one or more servers connected to the device via a network).

The plurality of selectable inputs may comprise a third selectable input 306 “ADD BLOCK TO SCENARIO”. Responsive to a selection of the third selectable input 306, one or more blocks may be selected and/or assigned to the scenario. For example, responsive to the selection of the third selectable input 306, a list of (e.g., previously configured) blocks may be displayed. In some examples, responsive to a selection of a block of the list of blocks, the block may be assigned to the scenario. In some examples, each block of the one or more blocks may correspond to a set of representations of (e.g., the plurality of representations of) the scenario. In some examples, an arrangement of the one or more blocks (e.g., and/or an order of presentation of one or more sets of representations corresponding to the one or more blocks) may be selected.

The plurality of selectable inputs may comprise a fourth selectable input 308 “ASSIGN SCENARIO TO USER”. Responsive to a selection of the fourth selectable input 308, a list of users may be displayed. In some examples, responsive to a selection of the user from the list of users, the user may be assigned to the scenario. Alternatively and/or additionally, responsive to the selection of the fourth selectable input 308, a username of the user may be inputted.

The plurality of selectable inputs may comprise a fifth selectable input 310 “BLOCK SETTINGS”. Responsive to a selection of the fifth selectable input 310, a set of representations may be selected and/or assigned to a block. For example, responsive to a selection of the fifth selectable input 310, a list of (e.g., previously configured) representations may be displayed. In some examples, responsive to a selection of a representation of the list of representations, the representation may be assigned to the block. In some examples, an arrangement of the set of representations (e.g., of the block) (e.g., and/or an order of presentation of representations of the set of representations) may be selected.

The plurality of selectable inputs may comprise a sixth selectable input 312 “REPRESENTATION SETTINGS”. Responsive to a selection of the sixth selectable input 312, the graphical user interface 300 may be controlled to display a second interface comprising a second plurality of selectable inputs corresponding to a plurality of parameters of a representation of the plurality of representations (e.g., of the scenario).

FIG. 3B illustrates the graphical user interface 300 being controlled (e.g., by a device of the system 301) to display the second interface comprising the second plurality of selectable inputs corresponding to the plurality of parameters of the representation. The second plurality of selectable inputs may comprise a seventh selectable input 318 “TITLE” corresponding to a second title of the representation. For example, responsive to a selection of the seventh selectable input 318, a second title name of the second title may be inputted. The second plurality of selectable inputs may comprise an eighth selectable input 320 “LIBRARY” corresponding to a library. For example, responsive to a selection of the eighth selectable input 320, a library name of the library may be inputted. The representation may (e.g., then) be stored in a directory corresponding to the library.

In some examples, the second plurality of selectable inputs may comprise a first set of selectable inputs 324 “FEEDBACK SETTINGS”. A first threshold “EXCELLENT THRESHOLD” may be inputted via the first set of selectable inputs 324. For example, a time error and/or a distance error of an attempt performed (e.g., by the user) in association with the representation may be considered at a first level (e.g., excellent, advanced, etc.) if the time error and/or the distance error are less than (e.g., or equal to) the first threshold. Alternatively and/or additionally, a second threshold “ALLOWED THRESHOLD” may be inputted via the first set of selectable inputs 324. For example, the time error and/or the distance error of the attempt may be considered at a second level (e.g., average, above-average, acceptable, etc.) if the time error and/or the distance error are greater than (e.g., or equal to) the first threshold and less than (e.g., or equal to) the second threshold. Alternatively and/or additionally, the time error and/or the distance error may be discarded if the time error and/or the distance error are greater than (e.g., or equal to) the second threshold.

A portion of errors of a plurality of errors (e.g., generated based upon a plurality of attempts corresponding to the plurality of representations) corresponding to a first feedback setting “NORMAL RANDOM FEEDBACK” may be inputted via the first set of selectable inputs 324. The portion of errors may be included in a report (e.g., generated based upon a plurality of attempts corresponding to the plurality of representations). For example, the portion of errors may comprise a percentage (e.g., and/or a fraction) corresponding to a percentage of errors of the plurality of errors to be included in the report. The portion of errors may be randomly selected (e.g., by the device) from the plurality of errors. A type of (e.g., random) selection “RANDOM FEEDBACK DISTRIBUTION” may be selected via the first set of selectable inputs 324. For example, responsive to a selection of a first type of random selection “NORMAL”, the portion of errors may be (e.g., randomly) selected from (e.g., all of) the plurality of errors. Alternatively and/or additionally, responsive to a selection of a second type of random selection “INCREASING”, the portion of errors may be (e.g., randomly) selected from an initial part of the plurality of errors (e.g., initial 20% of the plurality of errors, initial 30% of the plurality of errors, initial 50% of the plurality of errors, etc.). Alternatively and/or additionally, responsive to a selection of a third type of random selection “DECREASING”, the portion of errors may be selected from a last part of the plurality of errors (e.g., last 20% of the plurality of errors, last 30% of the plurality of errors, last 50% of the plurality of errors, etc.).

In some examples, the second plurality of selectable inputs may comprise a second set of selectable inputs 330 “DOMAIN FEEDBACK”. A lower limit of a domain “LOWER LIMIT” may be inputted via the second set of selectable inputs 330. For example, the time error and/or the distance error of the attempt may be discarded if the time error and/or the distance error are less than (e.g., or equal to) the lower limit. Alternatively and/or additionally, an upper limit of the domain “UPPER LIMIT” may be inputted via the second set of selectable inputs 330. For example, the time error and/or the distance error of the attempt may be discarded if the time error and/or the distance error are greater than (e.g., or equal to) the upper limit. In some examples, a domain setting “APPLY THE DOMAIN” may be selected via the second set of selectable inputs 330. For example, responsive to a selection of a first domain setting “NO”, the domain (e.g., the upper limit and/or the lower limit) may not be applied to the time error and/or the distance error. For example, the time error and/or the distance error may not be discarded if they are between the lower limit and the upper limit. Alternatively and/or additionally, responsive to a selection of a second domain setting “YES”, the domain may be applied to the time error and/or the distance error. For example, the time error and/or the distance error may be discarded if they are between the lower limit and the upper limit.

In some examples, the second plurality of selectable inputs may comprise a ninth selectable input 332 “BLOCK RESULT”. For example, a type of characteristic corresponding to a set of errors corresponding to a set of representations (e.g., comprising the representation) of a block may be selected via the ninth selectable input 332. For example, responsive to a selection of a first type of characteristic “AVERAGE”, an average of the set of errors (e.g., corresponding to the block) may be generated and included in the report. Alternatively and/or additionally, responsive to a selection of a second type of characteristic “SUMMARY”, a maximum, a minimum, a mode, a median, etc. of the set of errors may be generated and included in the report. Alternatively and/or additionally, responsive to a selection of a third type of characteristic “NONE”, characteristics associated with the block may not be included in the report.

In some examples, the second plurality of selectable inputs may comprise a third set of selectable inputs 326 “OBJECT COLOR” corresponding to a color of an object of the representation. For example, a red color setting “RED COLOR”, a green color setting “GREEN COLOR” and/or a blue color setting “BLUE COLOR” may be inputted. The color of the object may be generated based upon the red color setting, the green color setting and/or the blue color setting.

In some examples, the second plurality of selectable inputs may comprise a fourth set of selectable inputs 334 “REPRESENTATION SETTINGS”. A time delay setting “TIME DELAY” may be selected via the fourth set of selectable inputs 334. For example, a time delay may be implemented responsive to detection of the attempt (e.g., corresponding to the representation) and/or responsive to completion of the representation based upon the time delay setting. For example, responsive to detection of the attempt and/or responsive to completion of the representation, a second representation (e.g., proceeding the representation) may be displayed after a time corresponding to the time delay.

A second color of an endpoint “ENDPOINT COLOR” (e.g., corresponding to the representation) may be selected via the fourth set of selectable inputs 334. A third color of a background “BACKGROUND COLOR” (e.g., corresponding to the representation) may be selected via the fourth set of selectable inputs 334. A shape of the object “OBJECT SHAPE” may be selected via the fourth set of selectable inputs 334.

A data storage setting “SAVE INFORMATION” corresponding to the representation may be selected via the fourth set of selectable inputs 334. For example, responsive to a selection of a first data storage setting “YES”, the time error and/or the distance error may be stored in a database of attempts stored on the device (e.g., and/or one or more servers connected to the device via a network connection). Alternatively and/or additionally, responsive to a selection of a second data storage setting “NO”, the time error and/or the distance error may not be stored in the database of attempts and/or may be discarded.

A location of the endpoint “ENDPOINT FUNCTION” may be selected via the fourth set of selectable inputs 334. A transparency (e.g., and/or a visibility) setting of the object “TRANSPARENCY FUNCTION” may be selected via the fourth set of selectable inputs 334. In some examples, the transparency setting of the object may be selected such that the object has a first transparency during (e.g., presentation of) a first part of the representation and/or the object has a second transparency during (e.g., presentation of) a second part of the representation.

A sound setting “SOUND FUNCTION” may be selected via the fourth set of selectable inputs 334. For example, responsive to a selection of a first sound setting, a sound may be outputted (e.g., via a speaker) responsive to detection of the attempt. Alternatively and/or additionally, responsive to a selection of a second sound setting, a sound may be outputted during (e.g., and/or before) (e.g., presentation of) the representation (e.g., and/or before detection of the attempt). Alternatively and/or additionally, responsive to a selection of a third sound setting, a sound may not be outputted responsive to detection of the attempt and/or during (e.g., presentation of) the representation.

A duration setting “DURATION FUNCTION” may be selected via the fourth set of selectable inputs 334. The duration setting may correspond to a duration (e.g., of time) of the representation and/or a second duration of the object moving from a starting point to the endpoint. A second time delay setting “BEFORE TIME DELAY” may be selected via the fourth set of selectable inputs 334. For example, a second time delay may be implemented responsive to displaying the object at the starting point. For example, responsive to displaying the object at the starting point, the object may begin to move towards the endpoint after a second time corresponding to the second time delay.

A continuity setting “CONTINUITY FUNCTION” may be selected via the fourth set of selectable inputs 334. For example, the continuity setting may be selected such that the object moves continuously from the starting point to the endpoint. Alternatively and/or additionally, the continuity setting may be selected such that the object (e.g., discontinuously) moves from the starting point to the endpoint along a set of points corresponding to a path of the object. For example, the object may be displayed at a first point of the set of points. The object may (e.g., then) be displayed at a second point of the set of points. The object may (e.g., then) be displayed at a third point of the set of points. The object may be displayed at each point of the set of points (e.g., consecutively) until the object is displayed at the endpoint.

A stop movement setting “STOP MOVEMENT FUNCTION” may be selected via the fourth set of selectable inputs 334. For example, the stop movement setting may be selected such that the object may stop moving responsive to the object reaching the endpoint. Alternatively and/or additionally, the stop movement setting may be selected such that the object may continue moving when the object reaches the endpoint.

The second plurality of selectable inputs may comprise a tenth selectable input 322 “ADD FORMULA”. In some examples, responsive to a selection of the tenth selectable input 322 a formula of a path of motion of the object, a speed of motion of the object and/or an acceleration of motion of the object may be inputted. The second plurality of selectable inputs may comprise a fifth set of selectable inputs 328 “EQUATIONS OF MOTION”. The path of motion of the object, the speed of motion of the object and/or the acceleration of motion of the object may be configured via the fifth set of selectable inputs 328.

The second plurality of selectable inputs may comprise an eleventh selectable input 336 “ANIMATION”. For example, responsive to a selection of the eleventh selectable input 336 a simulation of the representation may be displayed via the second interface (e.g., and/or a third interface). The second plurality of selectable inputs may comprise a twelfth selectable input 338 “SAVE REPRESENTATION”. Responsive to a selection of the twelfth selectable input 338, the representation may be recorded and/or stored (e.g., under the second title name) in a database of representations. The database of representations may be stored on the device (e.g., and/or on one or more servers connected to the device via a network). Alternatively and/or additionally, the representation may be stored in the directory corresponding to the library.

In some examples, representations, such as the first representation, the second representation and/or the third representation illustrated in FIGS. 2A-2F, may be generated based upon a plurality of inputs received via the second interface illustrated in the FIG. 3B.

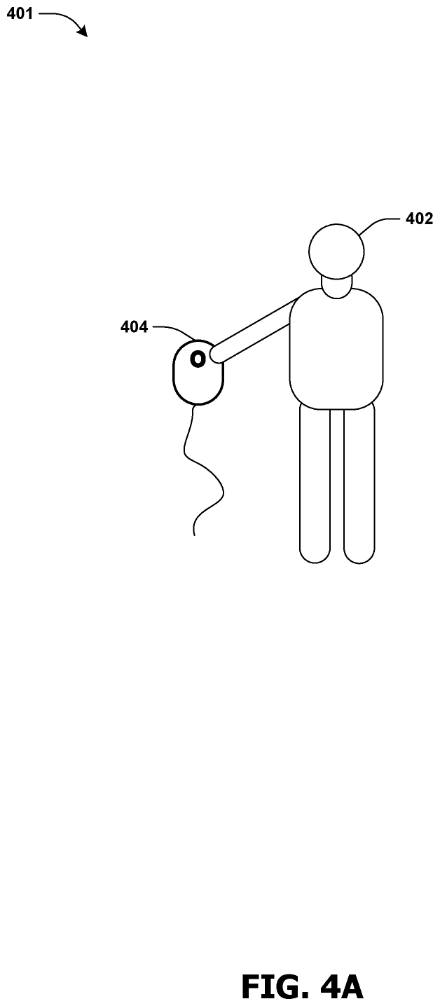

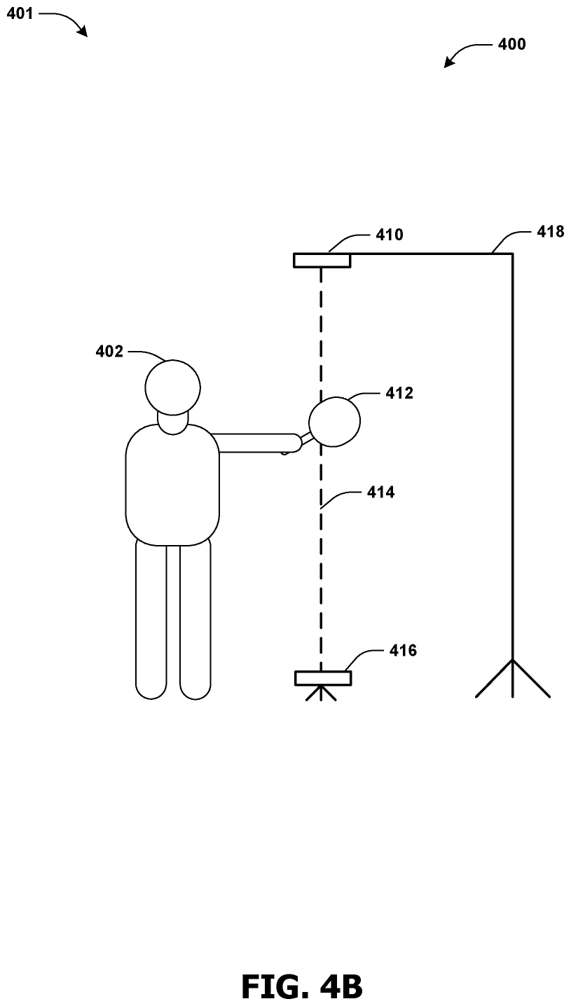

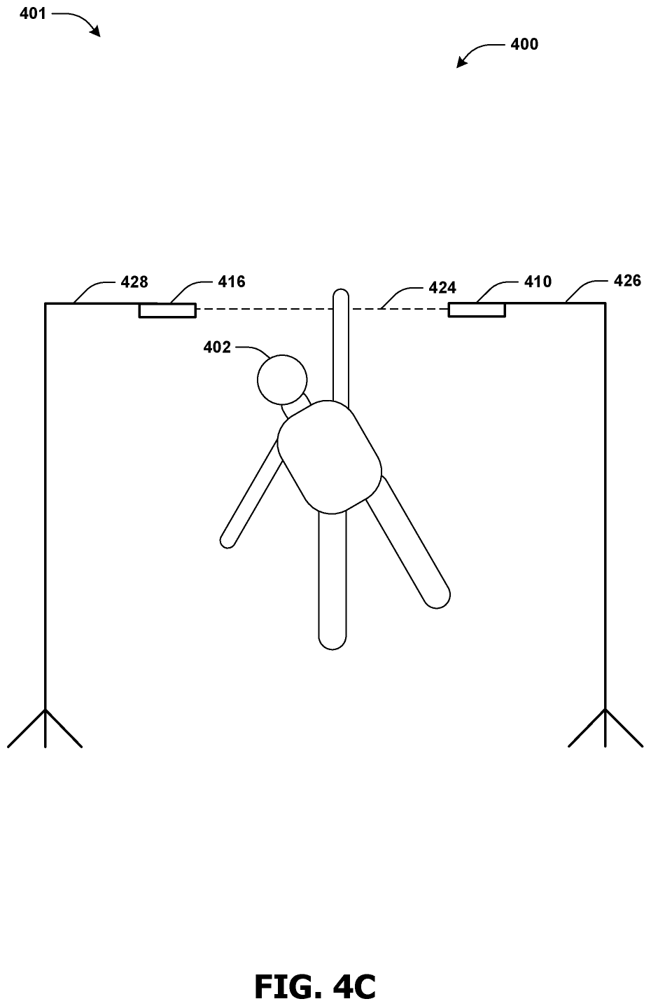

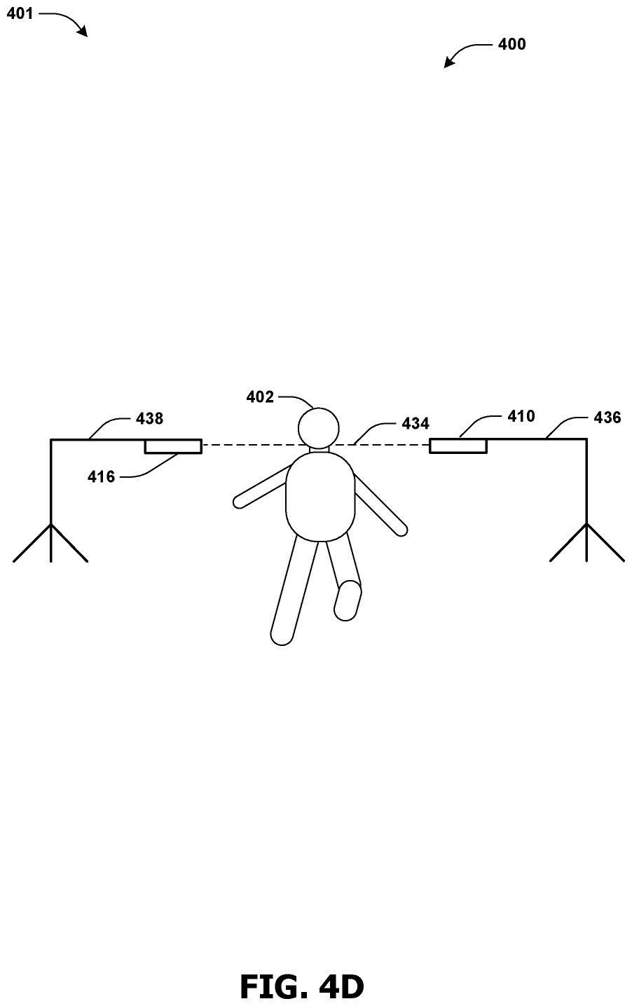

FIGS. 4A-4E illustrate examples of a system 401 for assessing coincidence-anticipation timing and/or motion perception of a user 402. For example, a screen may be controlled by a device to display an interface comprising a representation of an object moving from a starting point to an endpoint. The object may be configured to reach the endpoint at a first time. In some examples, an attempt (e.g., by the user 402) to activate a response device when the object reaches the endpoint may be monitored for (e.g., by the device). For example, the user 402 may be directed (e.g., and/or instructed) to activate the response device when the object reaches the endpoint.

In some examples, the representation may be a part of an assessment to determine a coincidence-anticipation timing ability and/or a motion perception ability of a user 402. Alternatively and/or additionally, the representation may be a part of a training activity to improve the coincidence-anticipation ability and/or the motion perception ability of the user 402. In some examples, the assessment and/or the training activity may be associated with a context. The context may comprise a sport (e.g., tennis, soccer, basketball, volleyball, table tennis, track, a combat sport, etc.) and/or an activity (e.g., driving, walking outdoors, performing household functions, etc.). In some examples, parameters of the representation (e.g., a path that the object moves along, a speed of movement of the object, etc.) may be configured based upon the context.

FIG. 4A illustrates the response device comprising a switch 404 (e.g., a pushbutton, an on-off switch, etc.). The user 402 may view the representation via the screen. The switch 404 may be coupled to the device via one or more (e.g., wireless and/or wired) connections. The switch 404 may be positioned adjacent to (e.g., in front of, above, below, to a side of, etc.) the user 402. The switch 404 may be configured to transmit the signal to the device responsive to activation of the switch 404. The signal may comprise an electronic signal (e.g., a pulse) and/or an electronic message indicating that the switch 404 is activated.

FIG. 4B illustrates the response device, configured for one or more first contexts, comprising a light transmitter 410 and/or a light sensor 416. The light transmitter 410 may be configured to emit light (e.g., a laser beam and/or a different type of light) through a first location 414. The light sensor 416 may be configured to monitor the light via the first location 414. In some examples, the light sensor 416 may detect motion at the first location 414. In some examples, the light sensor 416 may transmit the signal to the device responsive to detecting motion at the first location 414.

In some examples, the light transmitter 410 and/or the light sensor 416 may be positioned based upon the one or more first contexts. The light transmitter 410 may be positioned above the light sensor 416. For example, the light transmitter 410 may be coupled to a first mount 418. The first location 414 may be positioned adjacent to (e.g., in front of, to a side of, etc.) the user 402.

In some examples, the one or more first contexts may comprise table tennis, tennis, baseball, cricket, etc. For example, the user 402 may be directed to swing a sports object 412 (e.g., a tennis racket, a table tennis racket, a baseball bat, a cricket bat, etc.) through the first location 414 when the object reaches the endpoint. Alternatively and/or additionally, the one or more first contexts may comprise a combat sport. For example, the user 402 may be directed to swing a hand (e.g., and/or punch) through the first location 414 when the object reaches the endpoint. Alternatively and/or additionally, the one or more first contexts may comprise soccer, football, etc. For example, the user 402 may be directed to kick (e.g., a foot) through the first location 414 when the object reaches the endpoint.

FIG. 4C illustrates the response device, configured for one or more second contexts, comprising the light transmitter 410 and/or the light sensor 416. The light transmitter 410 may be configured to emit light (e.g., a laser beam and/or a different type of light) through a second location 424. The light sensor 416 may be configured to monitor the light via the second location 424. In some examples, the light sensor 416 may detect motion at the second location 424. In some examples, the light sensor 416 may transmit the signal to the device responsive to detecting motion at the second location 424.

In some examples, the light transmitter 410 and/or the light sensor 416 may be positioned based upon the one or more second contexts. The light transmitter 410 may be positioned across from the light sensor 416. For example, the light transmitter 410 may be coupled to a first mount 426. The light sensor 416 may be coupled to a second mount 428. A vertical distance from a floor (e.g., on which the user 402, the first mount 426 and/or the second mount 428 are positioned) of the light transmitter 410 (e.g., and/or a height of the first mount 426) may be the same as a vertical distance from the floor of the light sensor 416 (e.g., and/or a height of the second mount 428). The second location 424 may be positioned above the user 402.

In some examples, the one or more second contexts may comprise volleyball, lacrosse, tennis, etc. For example, the user 402 may be directed to swing a second object (e.g., a tennis racket, a lacrosse stick, etc.) through the second location 424 when the object reaches the endpoint. Alternatively and/or additionally, the user 402 may be directed to (e.g., vertically) swing a hand through the second location 424 (e.g., such as to perform a spike in volleyball) when the object reaches the endpoint.

FIG. 4D illustrates the response device, configured for one or more third contexts, comprising the light transmitter 410 and/or the light sensor 416. The light transmitter 410 may be configured to emit light (e.g., a laser beam and/or a different type of light) through a third location 434. The light sensor 416 may be configured to monitor the light via the third location 434. In some examples, the light sensor 416 may detect motion at the third location 434. In some examples, the light sensor 416 may transmit the signal to the device responsive to detecting motion at the third location 434.

In some examples, the light transmitter 410 and/or the light sensor 416 may be positioned based upon the one or more third contexts. The light transmitter 410 may be positioned across from the light sensor 416. For example, the light transmitter 410 may be coupled to a third mount 436. The light sensor 416 may be coupled to a fourth mount 438. A vertical distance from the floor of the light transmitter 410 (e.g., and/or a height of the third mount 436) may be the same as a vertical distance from the floor of the light sensor 416 (e.g., and/or a height of the second mount 428). The third location 434 may be positioned adjacent to (e.g., in front of) the user 402.

In some examples, the one or more third contexts may comprise track, sports related to running, combat sports, etc. For example, the user 402 may be directed to pass (e.g., a body of the user 402, a part of the body, etc.) through the third location 434 when the object reaches the endpoint. Alternatively and/or additionally, the user 402 may be directed to pass (e.g., a hand, a foot, etc.) through the third location 434 (e.g., such as to perform a punch, kick, etc.) when the object reaches the endpoint.

It may be appreciated that the disclosed subject matter may assist in performing an assessment to determine a coincidence-anticipation ability and/or a motion perception ability of a user and/or performing a training activity to improve the coincidence-anticipation ability and/or the motion perception ability of the user.

Implementation of at least some of the disclosed subject matter may lead to benefits including, but not limited to, an increase in accuracy and/or precision of the determining the coincidence-anticipation ability and/or the motion perception ability (e.g., as a result of a screen being controlled by a device to display an interface comprising a set of representations, consecutively, as a result of a first representation of the set of representations having first parameters, as a result of a second representation of the set of representations having second parameters, as a result of at least a portion of the first parameters being different from at least a portion of the second parameters, as a result of monitoring for a first attempt to activate a response device when a first object of the first representation reaches a first endpoint, as a result of detecting the first attempt at a first time by receiving a first signal from the response device, as a result of determining the first time accurately, as a result of determining a time error of the first attempt based upon the first time and/or a second time that the first object is configured to reach the first endpoint, as a result of determining a first location of the object corresponding to the first time accurately, as a result of determining a distance error of the first attempt based upon the first location and/or the first endpoint, as a result of determining a plurality of characteristics of a set of time errors and/or a set of distance errors corresponding to the set of representations, etc.).

Alternatively and/or additionally, implementation of at least some of the disclosed subject matter may lead to benefits including a reduction in size of the device and/or more efficient transportation of the device (e.g., as a result of the device controlling the screen to display the interface comprising the set of representations rather than having a second device comprising a plurality of lights as provided in some methods and/or techniques, as a result of transporting the device to a second location and connecting the device to a second screen rather than transporting the second device comprising the plurality of lights as provided in some methods and/or techniques, etc.).

Alternatively and/or additionally, implementation of at least some of the disclosed subject matter may lead to benefits including providing the assessment and/or the training activity in a variety of contexts and/or an increase in accuracy and/or precision of determining a second coincidence-anticipation ability and/or a second motion perception ability of the user associated with a context (e.g., as a result of the response device comprising a light transmitter and/or a light sensor, as a result of as a result of the light transmitter emitting light through a first location, as a result of the light sensor monitoring light via the first location and/or detecting motion at the first motion, as a result of positioning the light sensor and/or the light transmitter order to position the first location to emulate a context of the variety of contexts, as a result of configuring parameters of the set of representations based upon the context, etc.).

Alternatively and/or additionally, implementation of at least some of the disclosed subject matter may lead to benefits including providing an administrator with information to determine a coincidence-anticipation timing ability and/or a motion response ability of an athlete and/or determine a sports-related ability of the athlete based upon the coincidence-anticipation timing ability and/or the motion response ability of the athlete (e.g., as a result of the screen being controlled by the device to display the interface comprising a second set of representations, as a result of the second set of representations having parameters configured based upon the sport, as a result of generating a report representative of the coincidence-anticipation timing ability and/or the motion response ability of the athlete, as a result of a graphical user interface being controlled to display a second interface comprising the report, etc.).

In some examples, at least some of the disclosed subject matter may be implemented on a client device, and in some examples, at least some of the disclosed subject matter may be implemented on a server (e.g., hosting a service accessible via a network, such as the Internet).

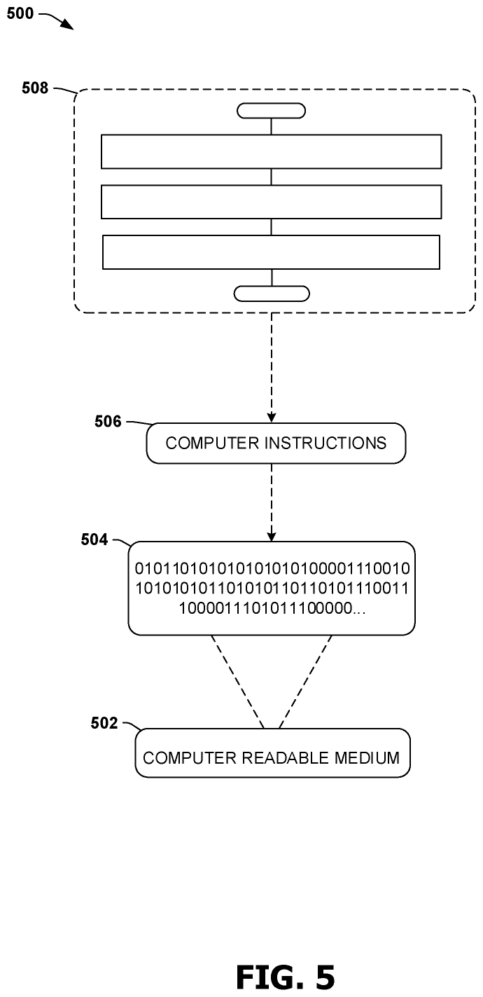

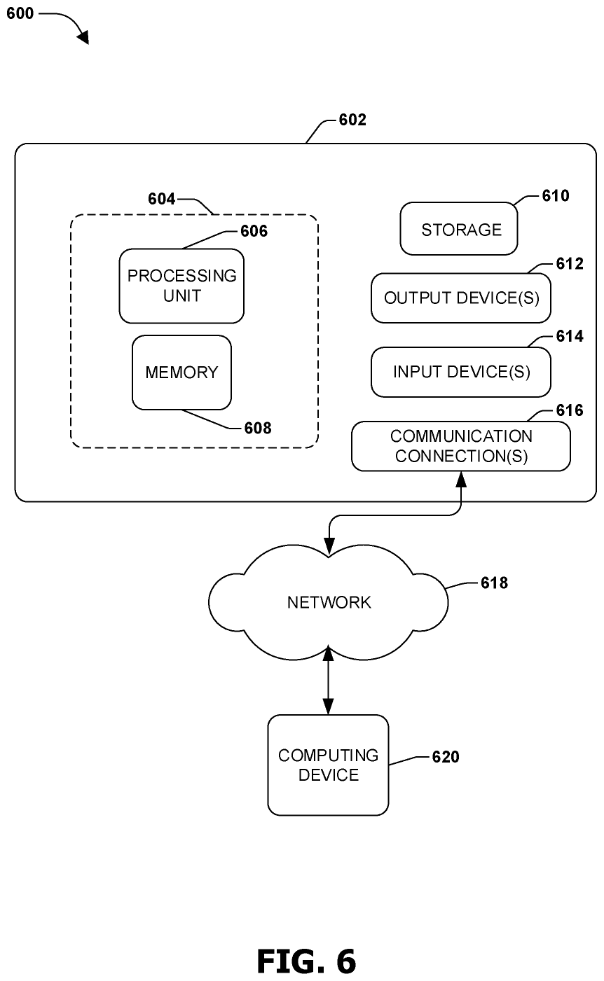

Another embodiment involves a computer-readable medium comprising processor-executable instructions. The processor-executable instructions may be configured to implement one or more of the techniques presented herein. An exemplary computer-readable medium that may be devised in these ways is illustrated in FIG. 5. An implementation 500 may comprise a computer-readable medium 502 (e.g., a CD, DVD, or at least a portion of a hard disk drive), which may comprise encoded computer-readable data 504. The computer-readable data 504 comprises a set of computer instructions 506 configured to operate according to one or more of the principles set forth herein. In one such embodiment 500, the processor-executable computer instructions 506 may be configured to perform a method, such as at least some of the exemplary method 100 of FIG. 1, for example. In another such embodiment, the processor-executable instructions 506 may be configured to implement a system, such as at least some of the exemplary system 201 of FIGS. 2A-2G, at least some of the exemplary system 301 of FIGS. 3A-3B, and/or at least some of the exemplary system 401 of FIGS. 4A-4D, for example. Many such computer-readable media 502 may be devised by those of ordinary skill in the art that are configured to operate in accordance with the techniques presented herein. FIG. 6 and the following discussion provide a description of a suitable computing environment to implement embodiments of one or more of the provisions set forth herein. The operating environment of FIG. 6 is just one example of a suitable operating environment and is not intended to indicate any limitation as to the scope of use or functionality of the operating environment. Example computing devices include, but are not limited to, server computers, mainframe computers, personal computers, hand-held or laptop devices, mobile devices (such as mobile phones, Personal Digital Assistants (PDAs), media players, and the like), consumer electronics, multiprocessor systems, mini computers, distributed computing environments that include any of the above systems or devices, and the like.

Although not required, embodiments are described in the general context of “computer readable instructions” being executed by one or more computing devices. Computer readable instructions may be distributed using computer readable media (discussed below). Computer readable instructions may be implemented as programs and/or program modules, such as functions, objects, Application Programming Interfaces (APIs), data structures, and the like, that execute particular tasks or implement particular abstract data types. Typically, the functionality of the computer readable instructions may be combined or distributed (e.g., as desired) in various environments.

FIG. 6 illustrates an example of a system 600 comprising a (e.g., computing) device 602. Device 602 may be configured to implement one or more embodiments provided herein. In an exemplary configuration, device 602 includes at least one processing unit 606 and at least one memory 608. Depending on the configuration and type of computing device, memory 608 may be volatile (such as RAM, for example), non-volatile (such as ROM, flash memory, etc., for example), or some combination of volatile and non-volatile. This configuration is illustrated in FIG. 6 by dashed line 604.

In other embodiments, device 602 may include additional features and/or functionality. For example, device 602 may further include additional storage (e.g., removable and/or non-removable) including, but not limited to, magnetic storage, optical storage, and the like. Such additional storage is illustrated in FIG. 6 by storage 610. In one embodiment, computer readable instructions to implement one or more embodiments provided herein may be in storage 610. Storage 610 may further store other computer readable instructions to implement an application program, an operating system, and the like. Computer readable instructions may be loaded in memory 608 for execution by processing unit 606, for example.

The term “computer readable media” as used herein includes computer storage media. Computer storage media includes volatile and/or nonvolatile, removable and/or non-removable media implemented in any method or technology for storage of information such as computer readable instructions or other data. Memory 608 and storage 610 are examples of computer storage media. Computer storage media may include, but is not limited to including, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which can be used to store desired information and can be accessed by device 602. Any such computer storage media may be part of device 602.

Device 602 may further include communication connection(s) 616 that allows device 602 to communicate with other devices. Communication connection(s) 616 may include, but is not limited to including, a modem, a radio frequency transmitter/receiver, an integrated network interface, a Network Interface Card (NIC), a USB connection, an infrared port, or other interfaces for connecting device 602 to other computing devices. Communication connection(s) 616 may include a wireless connection and/or a wired connection. Communication connection(s) 616 may transmit and/or receive communication media.

The term “computer readable media” may include, but is not limited to including, communication media. Communication media typically embodies computer readable instructions and/or other data in a “modulated data signal” such as a carrier wave or other transport mechanism and includes any information delivery media. The term “modulated data signal” may correspond to a signal that has one or more of its characteristics set or changed in such a manner as to encode information in the signal.

Device 602 may include input device(s) 614 such as mouse, keyboard, voice input device, pen, infrared cameras, touch input device, video input devices, and/or any other input device. Output device(s) 612 such as one or more displays, speakers, printers, and/or any other output device may also be included in device 602. Input device(s) 614 and output device(s) 612 may be connected to device 602 using a wireless connection, wired connection, or any combination thereof. In one embodiment, an input device or an output device from another computing device may be used as input device(s) 614 or output device(s) 612 for device 602.

Components of device 602 may be connected by various interconnects (e.g., such as a bus). Such interconnects may include a Peripheral Component Interconnect (PCI), such as a Universal Serial Bus (USB), PCI Express, an optical bus structure, firewire (IEEE 1394), and the like. In another embodiment, components of device 602 may be interconnected by a network. In an example, memory 608 may be comprised of multiple (e.g., physical) memory units located in different physical locations interconnected by a network.

Storage devices utilized to store computer readable instructions may be distributed across a network. For example, a computing device 620 accessible using a network 618 may store computer readable instructions to implement one or more embodiments provided herein. Device 602 may access computing device 620 and download a part or all of the computer readable instructions for execution. Alternatively, device 602 may download pieces of the computer readable instructions, as needed, or some instructions may be executed at device 602 and some at computing device 620.

Various operations of embodiments are provided herein. In one embodiment, one or more of the operations described may comprise computer readable instructions stored on one or more computer readable media, which if executed by a computing device, will cause the computing device to perform the operations described. The order in which some or all of the operations are described should not be construed as to imply that these operations are order dependent. Alternative ordering will be appreciated by one skilled in the art having the benefit of this description. Further, it will be understood that not all operations are present in each embodiment provided herein.

Although the subject matter has been described in language specific to structural features and/or methodological acts, it is to be understood that the subject matter defined in the appended claims is not limited to the specific features or acts described above. Rather, the specific features and acts described above are disclosed as example forms of implementing the claims.

As used in this application, the terms “system”, “component,” “interface”, “module,” and the like are generally intended to refer to a computer-related entity, either hardware, software, a combination of hardware and software, or software in execution. For example, a component may be, but is not limited to being, an object, a process running on a processor, a processor, a program, an executable, a thread of execution, and/or a computer. By way of illustration, an application running on a controller and the controller can be a component. One or more components may reside within a thread of execution and/or process and a component may be distributed between two or more computers and/or localized on one computer.

Furthermore, the claimed subject matter may be implemented as an apparatus, method, and/or article of manufacture using standard programming and/or engineering techniques to produce hardware, firmware, software, or any combination thereof to control a computer that may implement the disclosed subject matter. The term “article of manufacture” as used herein is intended to encompass a computer program (e.g., accessible from any computer-readable device, carrier, or media). Of course, those skilled in the art will recognize many modifications may be made to this configuration without departing from the scope or spirit of the claimed subject matter.

Moreover, the word “exemplary” is used herein to mean serving as an example, illustration, or instance. Any design or aspect described herein as “exemplary” is not necessarily to be construed as advantageous over other designs or aspects. Rather, use of the word “exemplary” is intended to present concepts in a concrete fashion. As used in this application, the word “or” is intended to mean an inclusive “or” (e.g., rather than an exclusive “or”). That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the words “a” and “an” as used in this application and the appended claims may generally be construed to mean “one or more” (e.g., unless specified otherwise or clear from context to be directed to a singular form). Also, at least one of A or B or the like generally means A or B or both A and B. Furthermore, to the extent that the terms “includes”, “having”, “has”, “with”, or variants thereof are used in either the detailed description or the claims, such terms are intended to be inclusive in a manner similar to the term “comprising.”

Although the disclosure has been shown and described with respect to one or more implementations, modifications and alterations will occur to others skilled in the art based (e.g., at least in part) upon a reading of this specification and the annexed drawings. The disclosure includes all such modifications and alterations. The disclosure is limited only by the scope of the following claims. In regard to the various functions performed by the above described components (e.g., resources, elements, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. Additionally, while a particular feature of the disclosure may have been disclosed with respect to only one of several implementations, the particular feature may be combined with one or more other features of the other implementations as may be desired and/or advantageous for any given or particular application.

What is claimed is:

1. A method for assessing at least one of coincidence-anticipation timing or motion perception of a user, the method comprising:
controlling a screen to display an interface comprising a first representation of a first object moving from a first starting point to a first endpoint, wherein the first object is configured to reach the first endpoint at a first time, wherein the first representation comprises:
the first object moving at a first speed;
the first object moving with a first acceleration; and
the first object moving in a first direction;
monitoring for a first attempt to activate a response device when the first object reaches the first endpoint;
detecting the first attempt at a second time, wherein the first attempt is detected by receiving a first signal from the response device;
controlling the screen to display the interface comprising a second representation of a second object moving from a second starting point to a second endpoint, wherein the second object is configured to reach the second endpoint at a third time, wherein the second representation comprises at least one of:
the second object moving at a second speed different from the first speed;
the second object moving with a second acceleration different from the first acceleration; or
the second object moving in a second direction different from the first direction;
monitoring for a second attempt to activate the response device when the second object reaches the second endpoint;
detecting the second attempt at a fourth time, wherein the second attempt is detected by receiving a second signal from the response device;
generating a first error of the first attempt based upon the first time and the second time;
generating a second error of the second attempt based upon the third time and the fourth time; and
generating a report comprising the first error and the second error, wherein the report is representative of a coincidence-anticipation timing ability of the user.

2. The method of claim 1, comprising:
controlling a graphical user interface to display a second interface comprising the report and one or more selectable inputs, wherein each selectable input of the one or more selectable inputs corresponds to a parameter of a plurality of parameters of a third representation, the plurality of parameters comprising at least one of:
a third speed of a third object of the third representation;
a third acceleration of the third object;
a third direction of movement of the third object;
a third starting point;
a third endpoint;
a shape of the third object;
a color of the third object; or
a color of a background of the interface; and
receiving, via the second interface, a request to present the third representation, wherein the request comprises one or more selections of the one or more selectable inputs corresponding to the plurality of parameters.

3. The method of claim 2, comprising:
controlling the screen to display the interface comprising the third representation of the third object moving from the third starting point to the third endpoint, wherein the third object is configured to reach the third endpoint at a fifth time, wherein the third representation comprises at least one of:
the third object moving at the third speed;
the third object moving with the third acceleration; or
the third object moving in the third direction;
monitoring for a third attempt to activate the response device when the third object reaches the third endpoint;
detecting the third attempt at a sixth time, wherein the third attempt is detected by receiving a third signal from the response device; and
generating a third error of the third attempt based upon the fifth time and the sixth time.

4. The method of claim 1, comprising:
generating a plurality of characteristics of a plurality of errors comprising the first error and the second error, wherein the plurality of characteristics comprises an average error of the plurality of errors, wherein the average error is representative of the combinational coincidence-anticipation timing ability of the user.

5. The method of claim 1, comprising:
receiving an input corresponding to a time delay between the first representation and the second representation; and
implementing the time delay between the first representation and the second representation.

6. The method of claim 1, comprising:
determining a first location of the first object at the second time;
generating a first distance error based upon the first location and the first endpoint;
determining a second location of the second object at the fourth time; and
generating a second distance error based upon the second location and the second endpoint, wherein the report comprises the first distance error and the second distance error.

7. The method of claim 6, comprising:
generating parameters of a third representation, wherein the parameters comprise at least one of a third speed of a third object of the third representation, a third acceleration of the third object, a third direction of movement of the third object, a third starting point or a third endpoint based upon at least one of the report, the first error, the second error, the first distance error or the second distance error.

8. The method of claim 7, comprising:
controlling the screen to display the interface comprising the third representation of the third object moving from the third starting point to the third endpoint, wherein the third object is configured to reach the third endpoint at a fifth time, wherein the third representation comprises at least one of:
the third object moving at the third speed;
the third object moving with the third acceleration; or
the third object moving in the third direction;
monitoring for a third attempt to activate the response device when the third object reaches the third endpoint;
detecting the third attempt at a sixth time, wherein the third attempt is detected by receiving a third signal from the response device; and
generating a third error based upon the fifth time and the sixth time.

9. A system for assessing at least one of coincidence-anticipation timing or motion perception of a user, comprising:
a computing device configured to:
control a screen to display an interface comprising a first representation of an object moving from a starting point to an endpoint, wherein the object is configured to reach the endpoint at a first time;
monitor for an attempt to activate a response device when the object reaches the endpoint;
detect the attempt at a second time, wherein the attempt is detected by receiving a signal from the response device;
generate an error of the attempt based upon the first time and the and the second time;
generate a report comprising the error, wherein the report is representative of a coincidence-anticipation timing ability of the user;
control a graphical user interface to display a second interface comprising the report and one or more selectable inputs, wherein each selectable input of the one or more selectable inputs corresponds to a parameter of a plurality of parameters of a second representation;
receive, via the second interface, a request to present the second representation, wherein the request comprises one or more selections of the one or more selectable inputs corresponding to the plurality of parameters; and
control the screen to display the interface comprising the second representation of a second object moving from a second starting point to a second endpoint.

10. The system of claim 9, the plurality of parameters comprising at least one of:
a speed of the second object;
an acceleration of the second object;
a direction of movement of the second object;
the second starting point;
the second endpoint;
a shape of the second object;
a color of the second object; or
a color of a background of the interface.

11. The system of claim 9, wherein the second object is configured to reach the second endpoint at a second time, the computing device configured to:
monitor for a second attempt to activate the response device when the second object reaches the second endpoint;
detect the second attempt at a fourth time, wherein the second attempt is detected by receiving a second signal from the response device; and
generate a second error of the second attempt based upon the third time and the fourth time.

12. The system of claim 11, the first representation comprising:
the object moving at a first speed;
the object moving with a first acceleration; and
the object moving in a first direction.

13. The system of claim 12, the second representation comprising at least one of:
the second object moving at a second speed different from the first speed;
the second object moving with a second acceleration different from the first acceleration; or
the second object moving in a second direction different from the first direction.

14. The system of claim 11, the computing device configured to:
receive an input corresponding to a time delay between the first representation and the second representation; and
implement the time delay between the first representation and the second representation.

15. The system of claim 9, the response device comprising a switch configured to transmit the signal responsive to activation of the switch.

16. The system of claim 9, the response device comprising:
a light transmitter configured to emit light through a first location; and
a light sensor configured to monitor the light via the first location.

17. The system of claim 16, the response device configured to transmit the signal responsive to detecting motion at the first location.

18. The system of claim 9, the computing device configured to:
receive a plurality of inputs corresponding to parameters of the representation, the parameters comprising at least one of:
a speed of the object;
an acceleration of the object;
a direction of movement of the object;
the starting point;
the endpoint;
a shape of the object;
a color of the object; or
a color of a background of the interface.

19. A non-transitory machine readable medium having stored thereon processor-executable instructions that when executed cause performance of operations for assessing at least one of coincidence-anticipation timing or motion perception of a user, the operations comprising:
controlling a screen to display an interface comprising a first representation of an object moving from a starting point to an endpoint, wherein the object is configured to reach the endpoint at a first time;
monitoring for an attempt to activate a response device when the object reaches the endpoint;
detecting the attempt at a second time, wherein the attempt is detected by receiving a signal from the response device;
generating an error of the attempt based upon the first time and the and the second time; and
generating a report comprising the error, wherein the report is representative of a coincidence-anticipation timing ability of the user.

20. The non-transitory machine readable medium of claim 19, the operations comprising:
determining a first location of the object at the second time; and
generating a first distance error based upon the first location and the first endpoint, wherein the report comprises the first distance error.
